(19) United States
(12) Patent Application Publication
(10) Pub. No.: US 2020/0261528 A1
(43) Pub. Date: Aug. 20, 2020

(54) PHARMACEUTICAL COMPOSITION FOR THE TREATMENT OF PROSTATIC HYPERPLASIA

(71) Applicant: PURYTRA FARMACEUTICI S.P.A., Milano (IT)

(72) Inventors: Guido PURICELLI, Milano (IT); Salvatore CUZZOCREA, Messina (IT); Elena SGARAVATTI, Padova (IT); Daniele PIETRA, Pontasserchio - San Giuliano Terme (IT)

(21) Appl. No.: 16/064,889

(22) PCT Filed: Dec. 21, 2016
(86) PCT No.: PCT/EP2016/082110

§ 371 Date: Jun. 21, 2018

(30) Foreign Application Priority Data
Dec. 21, 2015 (IT) 102015000085591

(51) Int. Cl. A61K 36/53; A61K 31/7024; A61K 31/7028; A61K 33/30; A61K 33/04; A61P 13/08; A61K 36/61

(52) U.S. Cl. CPC A61K 36/53; A61K 31/7024; A61K 31/7028; A61K 9/0053; A61K 33/04; A61P 13/08; A61K 36/61; A61K 33/30

(57) ABSTRACT

The present invention relates to a pharmaceutical composition comprising: —an extract of Ajuga reptans comprising an amount of phenylpropanoids from 30% to 70% by weight with respect to the weight of the extract, wherein said amount of phenylpropanoids comprises an amount from 10 to 50% of teupolioside with respect to the total phenylpropanoid weight, and —an extract of Epilobium comprising an amount from 20 to 50% by weight of polyphenols with respect to the weight of the extract, wherein said amount of polyphenols comprises up to 3% by weight of oenothein B with respect to the total amount of polyphenols. Also described is the pharmaceutical composition of the invention for use in the treatment of prostatic diseases, in particular the benign prostatic hyperplasia.

### FIELD OF THE INVENTION

The invention relates to a pharmaceutical composition comprising an extract of Ajuga with a quantity of phenylpropanoid from 30% to 70% in turn comprising an amount from 10 to 50% of teupolioside, and an Epilobium extract comprising a quantity from 20 to 50% of polyphenols in turn including an amount of oenothein B up to 3%. Preferably, said composition is suitable for use in the treatment of prostatic hyperplasia.

### State of the Art

The benign prostatic hyperplasia (BPH), also known as prostate adenoma (BEP—benign enlargement of the prostate) is a condition characterized by an increased volume of the prostate gland. The volume increase is due to hyperplasia (that is, an increase in the number of cells) of parenchymal and stromal component of the gland.

Many chemical mediators play a role in the symptoms and onset of BPH, such as androgens, estrogens, noradrenaline, acetylcholine, inflammatory cytokines. Among these, dihydrotestosterone (DHT) is the one of primary importance. DHT is a metabolite of testosterone and is a critical mediator of prostate growth. DHT is synthesized in the prostate from circulating testosterone. DHT is mainly localized in the cells of prostate connective tissue. Once synthesized, DHT reaches the epithelial cells. In both types of cells, DHT binds to the androgen receptor and signals the transcription of the growth factor to the mitogen of the two types of cells. The importance of DHT is supported by clinical observations of patients with benign hypertrophy, to which the administration of an inhibitor of 5α-reductase causes a significant reduction of DHT content in the prostate. In these patients, the volume of the prostate is reduced and consequently the symptoms of hypertrophy are reduced [See AUA guideline on management of benign prostatic hyperplasia (revisited 2010)].

The benign prostatic hyperplasia therapy is based on the use of different compounds: inhibitors of 5-alpha-reductase (5-ARI) such as dutasteride, finasteride; alpha blockers such as alfuzosin, doxazosin, tamsulosin, terazosin, silodosin; anticholinergic agents. Even combination therapies have been employed, such as alpha blocker along with inhibitor of 5-alpha-reductase, or also alpha blocker along with anticholinergics [AUA guideline on management of benign prostatic hyperplasia (revisited 2010)].

More beneficial effects can be obtained on benign prostatic hyperplasia with a nutraceutical approach (alone for mild cases and in combination with standard therapy for a moderate to severe disease).

Most used nutraceuticals include extracts of Serenoa repens, Pygeum africanum, Urtica dioica, Cucurbita pepo and substances such as isoflavones, lycopene, selenium and β-sitosterol (Phytother. Res. 2014 July; 28(7):949-55. Phytotherapy of benign prostatic hyperplasia. A minireview. Pagano E, Laudato M, Griffo M, Capasso R).

Furthermore, as treatment of benign prostatic hyperplasia also the use of Epilobium extracts is known, which contain oenothein B as active ingredient. (Granica S, Piwowarski J P, Czerwińska M E, Kiss A K. J. Ethnopharmacol. 2014 Oct. 28; 156:316-46; and Lesuisse D, Berjonneau J, Ciot C, Devaux P, Doucet B, Gourvest J F, Khemis B, Lang C, Legrand R, Lowinski M, Maquin P, Parent A, Schoot B, Teutsch G. J. Nat. Prod. 1996 May; 59(5):490-2).

An extract of Ajuga reptans, containing phenylpropanoids, including the teupolioside with title in the range from 20 to 90%, is described in EP1736166B1: as stated in this document, Ajuga reptans extracts have shown activity in patients with prostatic disease dependent on activation of the 5-alpha-reductase. However, the need to provide effective treatments for prostatic diseases is still felt, in particular against benign prostatic hyperplasia.

### SUMMARY OF THE INVENTION

The above object has been achieved by a pharmaceutical composition comprising:
an extract of Ajuga reptans comprising an amount of phenylpropanoids from 30% to 70% by weight with respect to the weight of the extract, wherein said amount of phenylpropanoids comprises from 10 to 50% of teupolioside with respect to the total phenylpropanoid weight, and
an extract of Epilobium comprising an amount from 20 to 50% by weight of polyphenols with respect to the weight of the extract, wherein said amount of polyphenols comprises up to 3% by weight of oenothein B with respect to the total amount of polyphenols.

The inventors of the present invention have surprisingly found that two specific extracts of Ajuga reptans and Epilobium have shown synergistic activity in the treatment of prostate diseases, in particular the benign prostatic hyperplasia. According to another aspect, therefore, the invention relates to a pharmaceutical composition comprising an extract of Ajuga reptans comprising an amount of phenylpropanoids from 30% to 70% in turn comprising 10 to 50% of teupolioside, and an Epilobium extract comprising 20 to 50% of polyphenols in turn comprising up to 3% of oenothein B for use in the treatment of prostatic diseases, in particular the benign prostatic hyperplasia.

### DESCRIPTION OF FIGURES

FIG. 1 is a graph relating to the weight of the prostate in rats treated as in Example 2;

FIG. 2 relates to the prostate histological aspects of rats treated as in Example 2;

FIG. 3 is a graph relative to the levels of PGE2 in the prostate in rats treated as in Example 2;

FIG. 4 is a graph relative to the levels of haematic DHT in the prostate of rats treated as in Example 2;

FIG. 5 shows the results of the evaluation of degradation of IκBα resulting in NFκB translocation into the nucleus as shown in Example 2;

FIG. 6 shows the results of the evaluation of iNOS and COX-2 expression as indicated in Example 2;

FIG. 7 shows the results of the evaluation of lipid peroxidation, malondialdehyde dosage as shown in Example 2;

FIG. 8 shows the results of the evaluation of apoptosis as indicated in Example 2;

FIG. 9 relates to the prostate histological aspects of rats treated as in Example 3;

FIG. 10 shows the effects of preparations of Example 3 in modulation of PGE2 and DHT levels;

FIG. 11 shows the effects of the preparations of Example 3 on expression of IκB-α and nuclear translocation of NF-κB;

FIG. 12 shows the effects of the preparations of Example 3 on expression of iNOS, COX-2 and MDA levels after BPH; and

FIG. 13 shows the effects of the preparations of Example 3 on the modulation of apoptosis pathways.

### DETAILED DESCRIPTION OF THE INVENTION

The present invention therefore relates to a pharmaceutical composition comprising:
an extract of Ajuga reptans comprising an amount of phenylpropanoids from 30% to 70% by weight with respect to the weight of the extract, wherein said amount of phenylpropanoids comprises from 10 to 50% of teupolioside with respect to the total phenylpropanoid weight, and
an extract of Epilobium comprising an amount from 20 to 50% by weight of polyphenols with respect to the weight of the extract, wherein said amount of polyphenols comprises up to 3% by weight of oenothein B with respect to the total amount of polyphenols.

In the present invention, when the term “phenylpropanoids” is used, it is intended compounds having the following general structure:

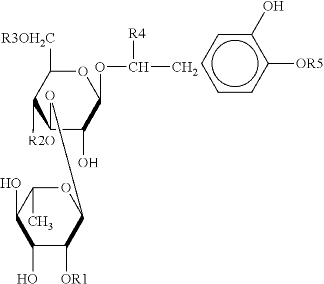

wherein R1 is a hydrogen atom or a monosaccharide with 5 or 6 carbon atoms or a disaccharide with 10 or 12 carbon atoms;
R2 is a hydrogen atom or a caffeoyl group (A) or feruloyl group;
R3 is a hydrogen atom or a monosaccharide with 5 or 6 carbon atoms or a disaccharide with 10 or 12 carbon atoms or a caffeoyl group (A) or feruloyl group (B);
R4 is a hydrogen atom, or a linear or branched alkyl group with 1 to 3 carbon atoms and preferably with 1 carbon atom or a hydroxyl;
R5 is a hydrogen atom, or a linear or branched alkyl group with 1 to 3 carbon atoms and preferably with 1 carbon atom;
provided that, if R3 is a caffeoyl group (A) or feruloyl group (B)

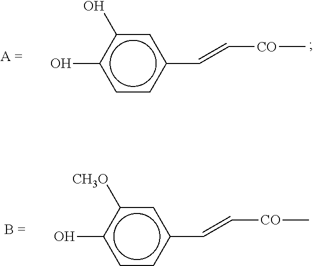

then R2 is a hydrogen atom and vice versa, R4 and R5 may both be the same or different from each other. In the family of the phenylpropanoids, teupolioside, isoteupolioside and methylisoteupolioside compounds are known. They have the following formulas:
Teupolioside:

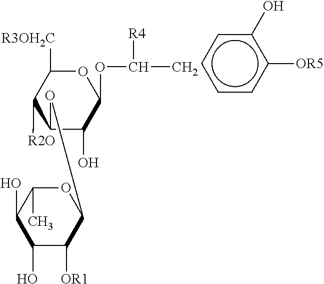

in which R1 is galactose, R2 is caffeic acid, R3 is hydrogen, R4 is hydrogen and R5 is hydrogen.
Methylteupolioside

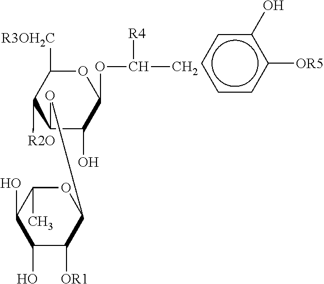

In which R1 is galactose, R2 is ferulic acid, R3 is hydrogen, R4 is hydrogen and R5 is hydrogen.
Isoteupolioside

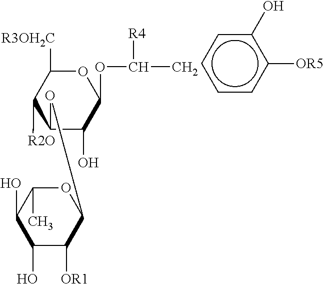

in which R1 is galactose, R2 is hydrogen, R3 is caffeic acid, R4 is hydrogen and R5 is hydrogen.

The pharmaceutical composition of the invention comprises an extract of Ajuga reptans comprising an amount of phenylpropanoids from 30% to 70% by weight with respect to the weight of the extract, said amount of phenylpropanoids comprising an amount of 10 to 50% by weight of teupolioside with respect to the phenylpropanoid amount.

Preferably, said extract of Ajuga reptans comprises an amount of phenylpropanoids of about 50% by weight with respect to the weight of the extract, more preferably in turn comprising about 30% by weight of teupolioside with respect to the phenylpropanoid amount.

According to the invention, in one preferred and advantageous embodiment, the extract of Ajuga reptans of the composition of the present invention is an extract sold with the commercial name of Teoside™50 by Istituto di Ricerche Biotecnologiche (IRB) S.p.A. (Altavilla Vicentina, Italy).

The extract of the preferred and advantageous embodiment of the invention comprises about 50% of phenylpropanoids with respect to the total weight of the extract, and said amount of phenylpropanoids includes about 30% of Teupolioside. Also methylteupolioside and isoteupolioside are present in said extract.

The composition of the invention also comprises an extract of Epilobium comprising polyphenols in an amount of 20 to 50% with respect to the weight of the extract, said amount of polyphenols in turn comprising up to 3% of oenothein B.

Oenothein B has the following formula:

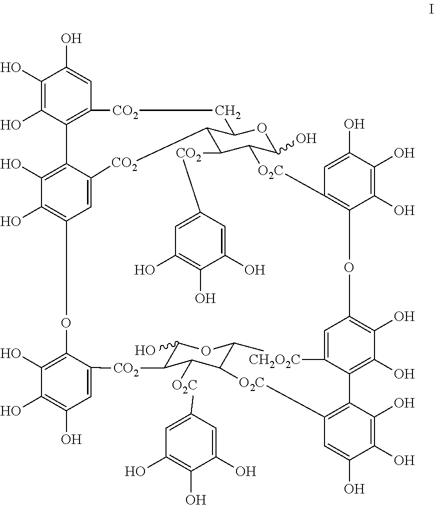

including its tautomers and structural conformers.

There are approximately 200 different species of Epilobium. Epilobium contains sterols, triterpenes, flavonoids and polyphenols.

There are therefore a number of Epilobium extracts that include a certain amount of polyphenols. Preferably, the extract of Epilobium of the invention is obtained from Epilobium angustifolium and/or parviflorum and comprises from 20 to 50% of polyphenols, in turn comprising up to 3% of oenothein B.

In a preferred and advantageous embodiment of the invention, the pharmaceutical composition comprises an extract of leaves and stems of 50% of Epilobium angustifolium and 50% of Epilobium parviflorum, said extract comprising from 30 to 33% by weight of polyphenols with respect to the weight of the extract, more preferably about 31.7%, in turn comprising up to 0.4% by weight of oenothein B, more preferably about 0.31% of oenothein B. The Epilobium extract of the invention is preferably obtained by extraction with hot water and then drying the aqueous solution (aqueous extract) so resulting, in order to obtain a dry extract comprising 20 to 50% of polyphenols, in turn comprising up to 3% of oenothein B.

In the preferred and advantageous embodiment of the invention, the Epilobium extract was obtained by extraction in hot water of a 50:50 mixture of the aerial parts of Epilobium angustifolium L. and Epilobium parviflorum Scherb.

The pharmaceutical composition of the invention therefore includes the above-mentioned extract of Ajuga reptans and the aforementioned Epilobium extract. Preferably, the pharmaceutical composition comprises a weight ratio from 1:1 to 1:20, more preferably from 1:1 to 1:10, even more preferably of 1:6 of Ajuga reptans extract to Epilobium extract.

The composition of the invention further comprises maltodextrins, such as maltodextrin obtained from corn, bulking agents such as dicalcium phosphate; zinc-L-pidolate, anti-caking agents, such as magnesium stearate, silicon dioxide; sodium selenite, in order to prepare a pharmaceutical preparation. The composition, and therefore, the pharmaceutical preparation according to the invention can be administered through any route of administration, preferably by oral administration, more preferably in the form of gelatin capsules.

Preferably, the composition of the invention is administered as a preparation, preferably oral, in the form of unit dosage, for example as a capsule or tablet, comprising 100 to 200 mg of Epilobium extract that comprises an amount of 20 to 50% by weight of polyphenols with respect to the weight of the extract, said amount of polyphenols in turn comprising up to 3% of oenothein B, and 10-40 mg of dry extract of Ajuga reptans comprising an amount of polypropanoids of 10-15 mg.

More preferably the composition of the invention is administered as a preparation in the form of unitary dose, preferably oral, for example as a capsule or tablet, comprising 150 mg of Epilobium extract that comprises an amount of 20 to 50% of polyphenols with respect to the weight of the extract, said amount of polyphenols in turn comprising up to 3% of oenothein B, and 25 mg of dry extract of Ajuga reptans comprising an amount of polypropanoids of 12.5 mg. Even more preferably, the composition of the invention is administered as a preparation in the form of unitary dose, preferably oral for example as a capsule or tablet, comprising 150 mg of Epilobium extract that comprises an amount of 20 to 50% of polyphenols with respect to the weight of the extract, said amount of polyphenols in turn comprising up to 3% of oenothein B, and 25 mg of dry extract of Ajuga reptans comprising an amount of polypropanoids of 12.5 mg, Zinc-L-pidolate equal to 3 mg of Zinc and sodium selenite equal to 27.5 μg of selenium.

In another aspect, the invention relates to a pharmaceutical composition comprising an extract of Ajuga reptans comprising an amount of phenylpropanoids from 30% to 70% with respect to the weight of the extract, in turn comprising an amount of 10 to 50% of teupolioside with respect to the total phenylpropanoid weight, and an Epilobium extract comprising an amount of 20 to 50% of polyphenols with respect to the weight of the extract, said amount of polyphenols in turn comprising up to 3% of oenothein B for use in the treatment of prostatic diseases, in particular the benign prostatic hyperplasia.

All the preferred and advantageous embodiments indicated above for the two extracts and for the pharmaceutical composition are the same for the use of the composition in the treatment of prostate diseases, in particular the benign prostatic hyperplasia.

Some exemplary embodiments of the invention and the evaluation of the synergy deriving from the composition of the invention, are given in the following, by way of example and not limitative of the invention itself.

### EXPERIMENTAL SECTION

### Example 1: Preparation of the Composition of the Invention

An extract of Epilobium was prepared by extraction in hot water of a 50:50 mixture of the aerial parts of Epilobium angustifolium L. and Epilobium parviflorum Scherb. The aqueous solution so obtained was dried, thus obtaining an extract of ¼ weight as compared to the initial plant material. Maltodextrin obtained from corn at 30% was added as excipient/auxiliary substance. The amount of polyphenols in the extract compared to the extract weight was about 31.7%, while the amount of oenothein B (expressed as gallic acid) was about 0.31% relative to the weight of all polyphenols. The amount of polyphenols was determined according to the technique defined by the European Pharmacopoeia 8th Edition, and the amount of oenothein B was determined by HPLC-MC-QTOF. As extract of Ajuga reptans a product sold by Istituto di Ricerche Biotecnologiche (IRB) S.p.A. (Altavilla Vicentina, Italy) with the commercial name of Teoside™50 was used. The extract of Teoside™50 comprises about 50% of phenylpropanoids with respect to the total weight of the extract, and said amount of phenylpropanoids includes about 30% of Teupolioside.

The two extracts were combined in a ratio extract of Epilobium/commercial Teoside™50 of 12:2.

### Example 2: Evaluation of the Pharmaceutical Composition According to the Invention in Benign Prostatic Hyperplasia

The evaluation was carried out on an experimental model of benign prostatic hyperplasia after 14 days of treatment through oral administration. The choice of the dose and administration mode (oral with gavage) was performed in order to identify the effects caused by the oral administration of the above compounds and to determine any adverse reactions. The experimental design was so set up as to allow identification of both physiological changes, haematological and anatomo-pathological changes. Toxic effects could possibly also be identified from appearance of these changes.

The evaluation was performed on Sprague-Dawley male rats from the Company Charles River Laboratory (Calco, Milan, Italy).

Rat is in fact the “preferred choice” animal species for studies on prostate hyperplasia. The effects of both the invention preparations and the comparison ones on benign prostatic hyperplasia in the rat were then evaluated.

At the beginning of the test, the animals were approximately 3 months old, with a body weight between 220 and 250 grams. Housing conditions were as follows: air-conditioned environment at 21°−23° C. and 55±10% RH, with 12-h light-dark cycles.

The animals were kept for 7 days in the indicated conditions and were subjected to the following veterinary inspections: body weight measurements, water and food consumption, feces and urine tests; necropsy examination (random) of 5% of the arrived animals.

The following experimental groups were prepared: 3 groups of 5 animals, housed in groups of 5, in makrolon cages with flat bottom of 425×266×180 mm ht containing dust-free litter (dust-free sawdust) and fed with pellets standard diets (MIL mice and rats) and watered with tap water ad libitum. The rats were treated with testosterone propionate diluted in corn oil in a 3 mg/kg dose in a volume of 100 μl administered subcutaneously each day.

The tested preparations were the following:
Epilobium extract obtained as in Example 1 (12 mg/kg) orally administered for 14 days;
Commercial extract of Serenoa sold by Farmalabor Ltd. (Canosa di Puglia, Italy) in combination with selenium, which is a commercial product known for the treatment of benign prostatic hyperplasia; Selenium (0.005 mg/kg)+Serenoa (28.5 mg/kg) administered orally for 14 days.
14 mg/kg of composition of the invention as obtained in Example 1, comprising Teoside™50 (2 mg/kg) and Epilobium extract (12 mg/kg), orally administered for 14 days.

The controls (Sham) were treated with a carrier for the entire duration of the experiment.

Volume, Mode and Frequency of Administration:

Subcutaneous administration of testosterone propionate diluted in corn oil was at a dose of 3 mg/kg in a volume of 100 μl and oral administration with gavage of various preparations at a dose of 2 mg/kg in a volume of 250 μl orally for 14 days (once a day).

The following measurements were performed on the animals of all experimental groups:
prostate weight;
histological examination of the prostate;
measurement of prostaglandin E2
assessment of blood DHT
evaluation of inflammatory mediators by western blot analysis

Histological examination of the prostate: fixation in 10% neutral formalin, paraffin embedding and staining the prostate with hematoxylin-eosin.

Measurement of prostaglandin E2 in haematic DHT: levels of prostaglandin E2 and DHT in the serum were measured using an ELISA KIT.

Results

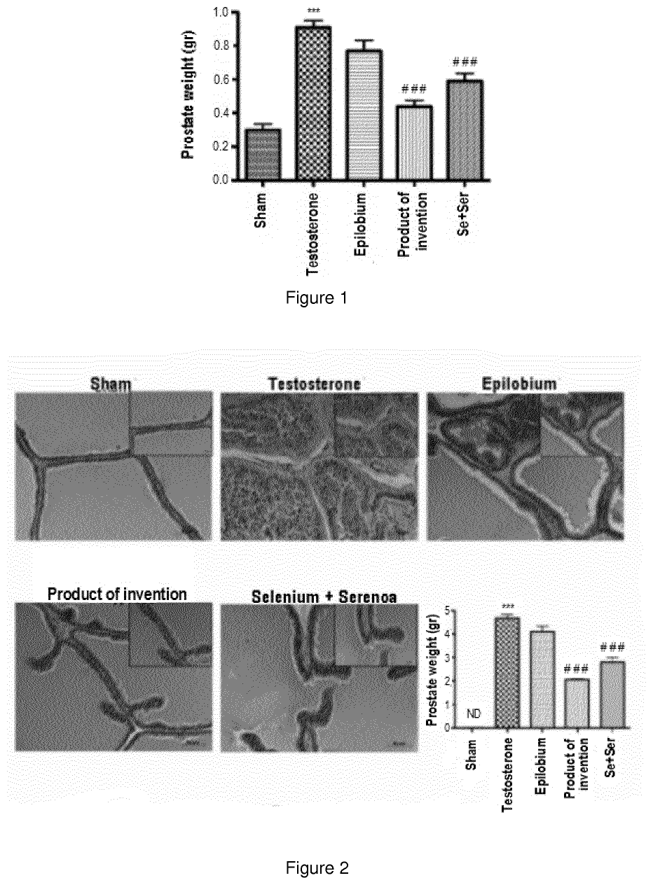

The investigations have shown that the preparation corresponding to the composition of the invention and the Selenium+Serenoa preparation were well tolerated by rats through oral administration for 14 consecutive days and did not cause any cases of death and/or significant change in body weight and/or significant change in food consumption, even in the case of benign prostatic hyperplasia. The study conducted further revealed that daily administration of testosterone induced a significant increase in prostate size. As is apparent from FIG. 1, the growth was decreased following administration of the Selenium+Serenoa preparation, but was further decreased by the treatment with the product of the invention.

Also an assessment was carried out, through the staining with hematoxylin and eosin, about prostate morphological alterations induced by daily administration of testosterone and the tested preparations. As is apparent from FIG. 2, the product of the invention reduced significantly, and more than the comparative Selenium+Serenoa preparation, histomorphological alterations of the prostate, restoring the regularity of the acini, the shape of the cells and the integrity of the basal membrane.

The reduction of the histological alterations of the same organ is also evident from the following Table 1.

TABLE 1

| | | | | Testosterone + | |
|---|---|---|---|---|---|
| | | | Testosterone + | product of the | Testosterone + |
| | Control | Testosterone | Epilobium | invention | Se + Ser |
| Regularity of the | Present | Absent | Absent | Present | Present |
| acini | | | | | |
| Stroma | Light | Variable | Variable | Light | Variable |
| Shape of | Cuboid/ | Cylindrical | Cylindrical | Cuboid/ | Cuboid/ |
| cells | cylindrical | | | cylindrical | cylindrical |
| Cellular polarity | Present | Absent | Absent/ | Present | Absent/ |
| | | | Present | | Present |
| Nuclear shape | Round | Round/ | Round/ | Round/ | Round/ |
| | | ovoid | ovoid | ovoid | ovoid |
| Mitosis | Absent | Present | Isolated | Absent | Isolated |
| Acinous villi | Absent | Light | Light | Light | Light |
| Cribriform | Absent | Absent | Absent | Absent | Absent |
| design | | | | | |
| Accumulations | Absent | Moderate | Isolated | Absent | Isolated |
| Growth | Absent | Moderate | Light | Absent | Absent |
| Basal | Intact | Intact/with | Intact/with | Intact | Intact |
| membrane | | interruptions | interruptions | | |

Morphological alterations of the prostate gland induced by daily administration of testosterone were evaluated through the hematoxylin and eosin staining. It has been observed that daily administration of testosterone induced a complete disorganization of the prostatic tissue and a marked hyperplasia. As summarized in Table 1 and shown in FIG. 2, following the daily treatment with testosterone an irregular shape of the acini with villi projections within the lumen and loss of epithelial polarity have been observed. The epithelium appeared to be cuboidal/cylindrical and nuclei were lined up irregularly. Again FIG. 2 and Table 1 show how the treatment with the product of the invention, reduced histomorphological alterations of the prostate, restored the regular shape of the acini, the epithelial polarity, the integrity of the basal membrane and reduced the villi projections into the lumen (Table 1 and FIG. 2).

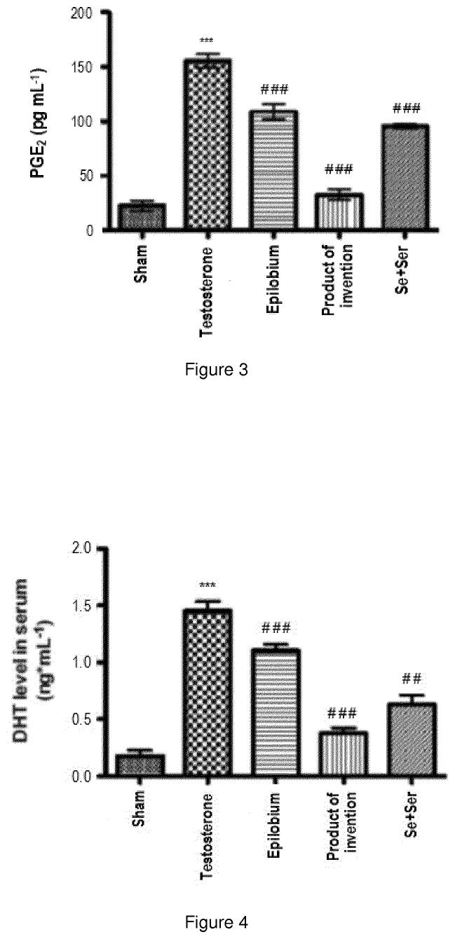

During the experiment the levels of prostaglandin E2 (PGE2) and blood dihydrotestosterone (DHT) were also assessed. As shown by FIG. 3 and FIG. 4, the preparation corresponding to the composition of the invention reduced the production of PGE2 and of DHT significantly and more effectively than the other compounds.

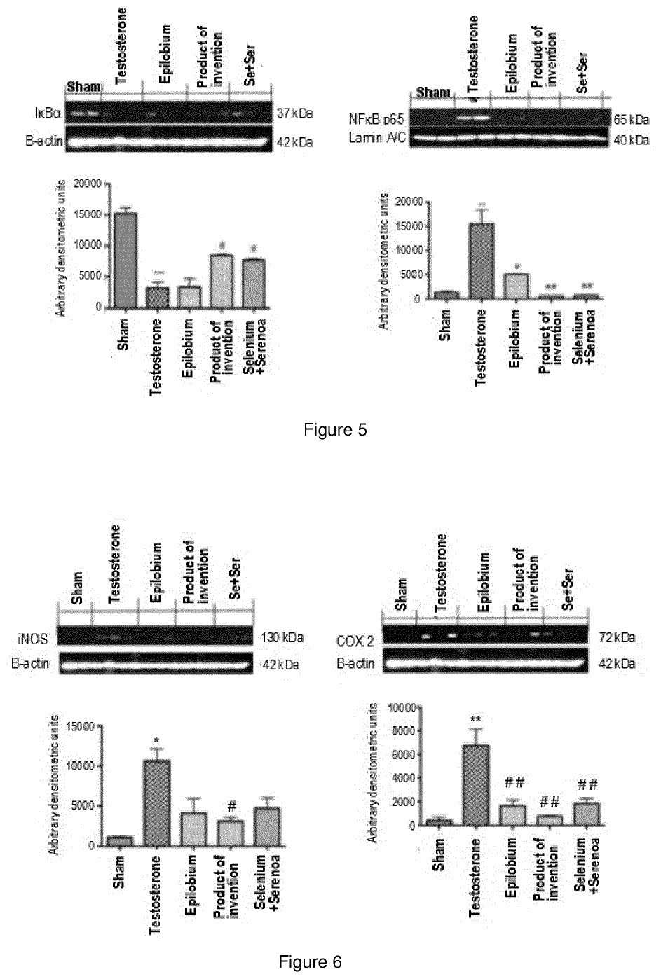

Also the onset of an inflammatory process following daily administration of testosterone was evaluated by western blot analysis. In particular, the levels of IκBα and NFκB were observed as well as the significant increase in the degradation of IκBα resulting in translocation of NFκB in the nucleus and induced by daily administration of testosterone. As is apparent from FIG. 5 the product of the invention could significantly reduce the degradation of IκBα, bringing it back to control levels, and the consequent translocation of NFκB into the nucleus.

Furthermore, the onset of oxidative stress and lipid peroxidation following the daily administration of testosterone was evaluated through the analysis of iNOS, COX-2 and the dosage of malondialdehyde.

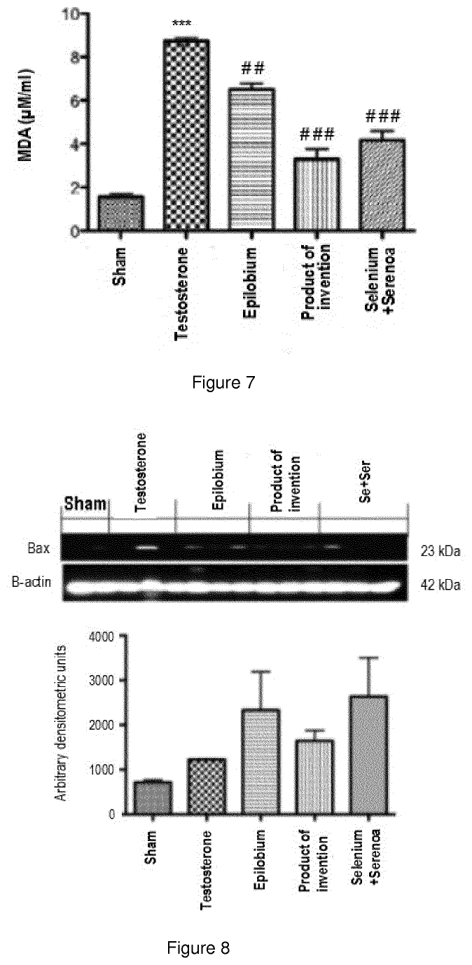

As reported in FIGS. 6 and 7, the results showed an increased expression of iNOS and COX-2 and an increase of lipid peroxidation following daily administration of testosterone, while treatment with the product of the invention is significantly reduced the increase of iNOS and COX-2 expression and lipid peroxidation.

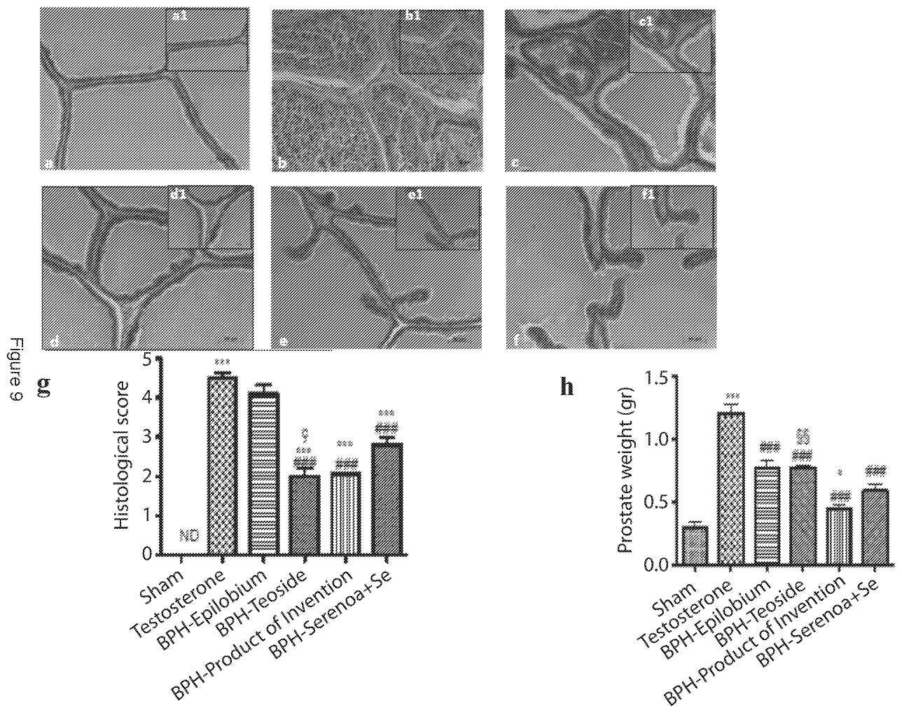

Finally, the onset of the apoptotic process following the daily administration of testosterone was assessed. As is apparent from FIG. 8, daily administration of testosterone significantly reduced the physiological process of apoptosis by reducing the levels of Bax, i.e. a pro-apototic protein. The treatment with the product of the invention restored the levels of Bax.

### Example 3: Comparison Evaluation of the Product of the Invention

In this study, the ability of the product of the invention was evaluated using an experimental model of BPH.

In particular animals were treated with the following products:
Epilobium: an Epilobium extract obtained as in Example 1 (12 mg/kg) was used;
TEOSIDE™50: an extract of Ajuga reptans as in Example 1 was used. The extract of Teoside™50 comprises about 50% of phenylpropanoids with respect to the total weight of the extract, and said amount of phenylpropanoids includes about 30% of Teupolioside.
The product of the invention was prepared as in Example 1 and comprised Teoside™50 and Epilobium extract; and
Commercial extract of Serenoa as described in Example 2 in combination with selenium, abbreviated as “SeR+Se”, which is a commercial product known for the treatment of benign prostatic hyperplasia.

The aim of the study was to assess the therapeutic effects of the products and to validate their efficacy as anti-oxidant and anti-inflammatory drugs.

Male Sprague-Dawley rats weighing approximately 200 g were obtained from Envigo (Italy) and were used in the study.

All animal care and experimental procedures complied with the standards for care and use of animal subjects as stated in the Guide for the Care and Use of Laboratory Animals (Institute of Laboratory Animal Resources, National Academy of Sciences, Bethesda, Md.) and were approved by the Ethics Committee of the University of Messina.

The In vivo experiment consisted in benign prostatic hyperplasia (BPH) induction Specifically, after 1 week of acclimation to the laboratory environment, animals were randomly assigned to the following groups:

Sham group: rats were treated with vehicle (100 mL corn oil s.c.);

BPH-group: BPH was induced in rats with testosterone propionate administration at the dose of 3 mg/kg, s.c. diluted in corn oil in a volume of 100 μl and administered for 14 days;

BPH-Teoside group: rats were treated with testosterone propionate as previously described and received a treatment with TEOSIDE™50 (2 mg/kg in a volume of 250 μl) orally every day until for 14 days;

BPH-Epilobium group: rats were treated with testosterone propionate as previously described and received a treatment with Epilobium (12 mg/kg) orally every day until for 14 days;

BPH-product of invention group: rats were treated with testosterone propionate as previously described and received a treatment with the product of Example 1 (14 mg/kg containing Teoside 50% 2 mg/kg and Epilobium 12 mg/kg) orally every day until for 14 days;

BPH-SeR+Se group: rats were treated with testosterone propionate as previously described and received a treatment with SeR (28.5 mg/kg) and Se (0.005 mg/kg) orally every day until for 14 days.

At the end of the experiment, animals were killed under anaesthesia, and their prostates were immediately removed and used for further analysis.

For TEOSIDE™50 (IRB) and testosterone propionate (Sigma-Aldrich, Milan, Italy) the doses and routes of administration were chosen according to previously published reports (Altavilla D, Minutoli L, Polito F, et al. Effects of flavocoxid, a dual inhibitor of COX and 5-lipoxygenase enzymes, on benign prostatic hyperplasia. British journal of pharmacology. 2012; 167:95-108).

Prostates will be removed and weighed, percentage of growth inhibition was calculated as follows: 100−[(TG (treated group)−Sham)/(BPH−Sham)×100], where TG are the values of the treated groups.

The tissue sections were stained with hematoxylin/eosin (H&E) and studied using light microscopy connected to an Imaging system (AxioVision, Zeiss, Milan, Italy) as previously described (Paterniti I, Impellizzeri D, Di Paola R, et al. Docosahexaenoic acid attenuates the early inflammatory response following spinal cord injury in mice: in-vivo and in-vitro studies. Journal of neuroinflammation. 2014; 11:6) for histology studies.

For the Western blot analysis, cytosolic and nuclear extracts were prepared with slight modifications of a published procedure (Cordaro M, Paterniti I, Siracusa R, Impellizzeri D, Esposito E, Cuzzocrea S. KU0063794, a Dual mTORC1 and mTORC2 Inhibitor, Reduces Neural Tissue Damage and Locomotor Impairment After Spinal Cord Injury in Mice. Molecular neurobiology. 2016). The levels of IκBα, iNOS, COX-2, Bax, Bcl2 were quantified in cytosolic fractions. Nuclear factor κB (NF-κB) p65 was quantified in nuclear fractions. Abs anti-IκBα (1:500, Santa Cruz Biotechnology) and with anti-iNOS (1:1000, BD-transduction), anti-COX-2 (1:1000, cell signaling,), and anti-BAX (1:500, Santa Cruz Biotechnology), anti-NF-κB p65 (1:500; cell signaling), anti-Bcl-2 antibody (1:500; Santa Cruz Biotechnology). Relative expression of protein bands was quantified by densitometry with BIORAD ChemiDoc™ XRS+software and standardized to β-actin levels. Images of blot signals (8 bit/600 dpi resolution) were imported to analysis software (Image Quant TL, v2003).

Malondialdehyde (MDA) levels in the prostate samples were determined as an indicator of lipid peroxidation as previously described (Paterniti I, Genovese T, Mazzon E, et al. Liver X receptor agonist treatment regulates inflammatory response after spinal cord trauma. Journal of neurochemistry. 2010; 112:611-624).

An evaluation of PGE2 in prostate tissue was carried out. Prostate samples were then homogenized and assayed for PGE2 using a available Cayman EIA kit (Cayman, Arcore, Milan, Italy).

An evaluation of Dihydrotestosterone (DHT) levels was also carried out. DHT levels were assayed in plasma samples with a commercially available ELISA kit (My BioSource).

All other reagents and compounds used were obtained from Sigma Chemical Company (Milan, Italy).

All values in the figures and in the following were expressed as mean±standard error of the mean (SEM) of N observations. For the in vivo studies N represented the number of animals studied. In the experiments involving histology the figures shown were representative of at least three experiments performed on different experimental days. The results were analysed by one-way ANOVA followed by a Bonferroni post-hoc test for multiple comparisons. A p-value of less than 0.05 was considered significant.

Results

Prostate Histology

In FIG. 9 the effect of the above preparations were reported.

Sham prostate from vehicle-treated rats showing regular acini with cuboidal and low cylindrical epithelium with round nuclei showing basal alignment (a,a1). BPH prostate showing irregular acinar shape with papillary projection into the lumen and foci of piling-up hyperplastic nodules were evident. The epithelium was high cylindrical, multilayered and round/ovoid nuclei were irregularly aligned (b,b1). Treatments with Epilobium, Teoside and SeR+Se (c,c1, d,d1 and f,f1 respectively, see densitometry g) decreased the histological pattern and the marked hyperplasia.

FIG. 9g was representative of at least three experiments performed on different experimental days. ***P<0.0001 Testosterone vs sham groups; ### P<0.0001 Epilobium, product of the invention, Se+Ser, Teoside vs Testosterone; °°° P<0.0001 product of the invention, Se+Ser, Teoside vs Epilobium; [symbol] P<0.05 Teoside vs Se+Ser (performed with two way ANOVA and Bonferroni test). Moreover, a significant increase of the prostate weight was observed in BPH-treated rats (h) while treatments with Epilobium, Teoside and SeR+Se reduced a grow of prostate (h).

FIG. 9h is representative of at least three experiments performed on different experimental days ***P<0.0001 Testosterone vs sham groups; ### P<0.0001 Epilobium, product of the invention, Se+Ser, Teoside vs Testosterone; ° P<0.05, product of the invention vs Epilobium; §§ P<0.001 Teoside vs product of the invention.

Prostate tissue collected from control group showed a normal prostate architecture and histology (FIGS. 9 and a1, see densitometry g), while a complete derangement of prostate tissue, a profound hyperplasia and significant inflammation were observed after BPH induction (FIGS. 9b and b1, see densitometry g).

Epilobium, Teoside and SeR+Se treatments (FIG. 9 c,c1, d,d1 and f,f1 respectively, see densitometry g) decreased the marked hyperplasia induced by testosterone administration. Instead a greater trend of protection (FIGS. 9e and e1, see densitometry g) was observed in the group treated with the product of the invention.

In addition, an increase of the prostate weight in BPH-treated rats (FIG. 9 h) was observed, whereas treatments with Epilobium, Teoside and SeR+Se significantly reduced overweight and grow of prostate (FIG. 9h). A greater reduction in prostate grow rate was observed after the treatment with product of the invention (FIG. 9h).

PGE2 and DHT Levels after BPH

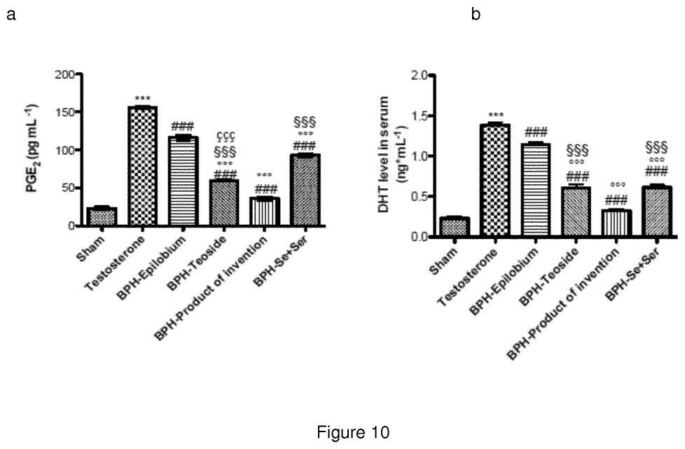

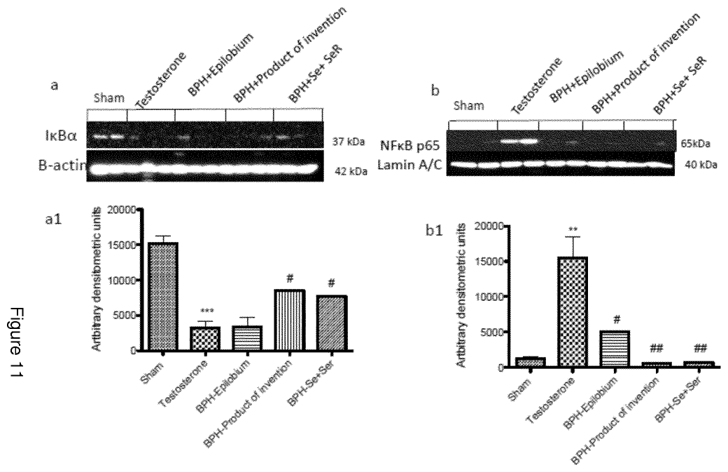

In FIG. 10 the effects of the above preparations in modulation of PGE2 and DHT levels are reported.

Basal levels of PGE2 and DHT were observed in the prostates samples collected from Sham animals (10a and b respectively). Testosterone administration determinated a marked increase in PGE2 and DHT levels (10a and b respectively), whereas treatment with Teoside and SeR+Se significantly reduced the increased levels in PGE2 (19a and b respectively).

The results were expressed as means±SD of 20 animals for each group. PGE2 levels ***P<0.0001 Testosterone vs sham groups; ### P<0.0001 Epilobium, product of the invention, Se+Ser, Teoside vs Testosterone; °°° P<0.0001 product of the invention, Se+Ser, Teoside vs Epilobium; § § § P<0.0001 Se+Ser, Teoside vs product of the invention; [symbol] çç P<0.0001 Teoside vs Se+Ser Homogenates of the prostates tissue from sham animals had basal levels of PGE2 (FIG. 10a). Prostates collected from BPH animals injected with testosterone showed a marked increase in PGE2 levels (FIG. 10a). Treatment with Teoside and SeR+Se significantly reduced the increased levels in PGE2 (FIG. 10a). A more pronounced trend of protection was observed in the group treated with product of the invention (FIG. 10a).

Another important parameter in current BPH treatment is the levels of dihydrotestosterone (DHT). In the present study basal levels of DHT in control group were observed; whereas a significantly increase of DHT levels was observed in BPH-rats (FIG. 10b).

Treatments with Epilobium, Teoside and SeR+Se considerably reduced DHT levels (FIG. 10b) and a better protection was observed with product of the invention administration compared to Teoside and SeR+Se (FIG. 10b).

IκBα and NFκB Expressions after BPH

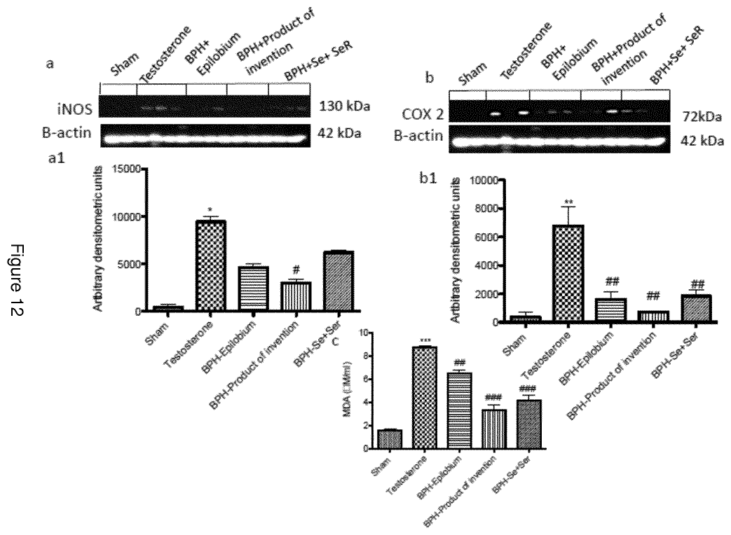

In FIG. 11 the effects of the above preparations on IκBα and NFκB expressions after BPH were evaluated.

Representative western blots showing the effects of Epilobium, the product of the invention, Teoside and SeR+Se on IκB-α degradation (A) and NF-κB p65 translocation (B), after testosterone inducing BPH were obtained.

A representative blot of lysates obtained from 20 animals/group was shown, and densitometry analysis of all animals was reported. The results in A and B are expressed as means±SD of 20 animals for each group. **P<0.01 vs Sham group, ***P<0.001 vs Sham group; ## P<0.01 vs BPH group; # P<0.05 vs BPH group.

To better evaluate the involvement of inflammatory process in the degree of BPH, hence the expression of IκBα and NFκB was analyzed. Basal levels of IκBα in control group were observed, whereas after BPH induction there was a significantly reduction of IκBα levels with a consequent activation/translocation of NFκB in the nucleus as showed in FIG. 11 a,a1 and b,b1. Treatment with the product of the invention and SeR+Se significantly prevented IκB-α degradation (FIG. 11 a,a1 and b,b1) and considerably reduced translocation of NFκB to the nucleus (FIG. 11 a,a1 and b,b1) preventing subsequent transcription of pro-inflammatory genes. No protection was observed with Epilobium treatment (FIG. 11 a,a1 and b,b1).

Induction of iNOS and COX-2 Expression after BPH

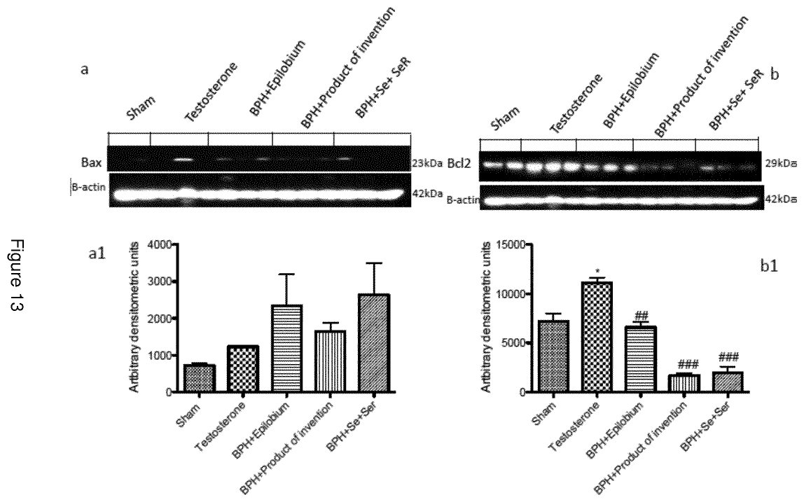

To understand the role of oxidative stress during BPH the levels of iNOS and COX-2 expression were analyzed. The results are reported in FIG. 12.

Representative western blots showing the effects of Epilobium, product of the invention, Teoside and SeR+Se treatments on iNOS (A), Cox-2 (B) levels after testosterone administration were obtained. Moreover, MDA levels were significantly reduced after Epilobium, product of the invention, Teoside and SeR+Se treatments (c).

A representative blot of lysates obtained from 20 animals/group is shown, and densitometry analysis of all animals is reported. The results in A, B and C were expressed as means±SD of 20 animals for each group. *P<0.005 vs Sham group; **P<0.01 vs Sham group; ***P<0.001 vs Sham group; ## P<0.01 vs BPH group; ### P<0.001 vs BPH group.

A significantly increase of both iNOS and COX-2 expression after BPH induction (FIG. 12 a,a1 and b,b1) was observed; whereas treatments with the product of the invention and SeR+Se demonstrated a significantly reduction of iNOS and COX-2 levels (FIG. 12 a,a1 and b,b1) confirming their anti-oxidant efficacy. Moreover, the extent of lipid peroxidation induced by oxidative stress, measured by MDA levels, were evaluated. An increase in MDA levels in BPH-group (FIG. 12 c) was observed; whereas product of the invention and SeR+Se treatments significantly protected from lipid peroxidation, reducing MDA levels as show in FIG. 12c. No protection was observed with Epilobium treatment (FIG. 12c).

Apoptosis Process in BPH

In FIG. 13 the effects of the above preparations on the modulation of apoptosis pathways are reported.

Specifically, representative western blots showing the effects of Epilobium, the product of the invention, Teoside and SeR+Se on Bax (A) and Bcl-2 (B) expression, after testosterone inducing BPH were obtained.

A representative blot of lysates obtained from 20 animals/group is shown, and densitometry analysis of all animals is reported. The results in A, B and C are expressed as means±SD of 20 animals for each group. *P<0.05 vs Sham group; ## P<0.01 vs BPH group; ### P<0.001 vs BPH group.

To evaluate how BPH can modulate apoptosis process both pro-apoptotic and anti-apoptotic proteins such as Bax and Bcl2 were observed.

The results obtained revealed a detectable levels of Bax and Bcl-2 in prostates tissue of the control group (FIG. 13 a,a1 and b,b1). Prostate tissue from BPH rats showed a slight increase in Bcl-2 level without changes in expression of Bax (FIG. 13 a,a1 and b,b1).

Treatments with the product of the invention and SeR+Se significantly reduced levels of anti-apoptotic Bcl-2 and increased expression of pro-apoptotic Bax (FIG. 13 a,a1 and b,b1).

In the present study it was clearly demonstrated that treatment with the product of the invention and SeR+Se caused a greater anti-inflammatory effect reducing degradation of IκBα and the translocation of NF-κB induced by 14 days of testosterone administration. Activation of NF-κB lead to an increased levels of pro-inflammatory genes such as pro-inflammatory cytokines (TNFα and IL-1β) as well as pro-inflammatory enzymes as iNOS and COX-2.

It seems that through the several inflammatory pathways, aberrant arachidonic acid (AA) metabolism with elevated COX-2 expression and increased levels of PGE2 has been linked with the development of BPH (Larre S, Tran N, Fan C, et al. PGE2 and LTB4 tissue levels in benign and cancerous prostates. Prostaglandins & other lipid mediators. 2008; 87:14-19). Several mechanisms have been proposed to explain the role of COX-2 in prostate over-growth, some of these effects may result from the COX-2-mediated increases in PG synthesis, especially of PGE2 (Larre S, Tran N, Fan C, et al. PGE2 and LTB4 tissue levels in benign and cancerous prostates. Prostaglandins & other lipid mediators. 2008; 87:14-19).

Thus, the results of this study demonstrated that after BPH induction there was a higher expressions of iNOS and COX-2; whereas treatments with SeR+Se and Epilobium significantly reduced the higher levels of both iNOS and COX-2 and the lipidic peroxidation that were generated after BPH. Moreover, it was shown that the product of the invention was the most effective treatment in blunting iNOS and COX-2 as well as MDA production.

It has also been demonstrated that COX-2 up-regulates the expression of anti-apoptotic protein Bcl-2 with a concomitant decrease in prostate tissue apoptosis; Bcl-2 and Bax are key players in apoptotic events, most cancers including prostate cancer, generally over-express Bcl-2 (Revelos K, Petraki C, Gregorakis A, Scorilas A, Papanastasiou P, Koutsilieris M. Immunohistochemical expression of Bcl2 is an independent predictor of time-to-biochemical failure in patients with clinically localized prostate cancer following radical prostatectomy. Anticancer research. 2005; 25:3123-3133) that neutralize apoptotic effect of Bax, thereby escaping apoptosis and reducing therapeutic efficiency; the ratio of these proteins determines whether a cell will undergo apoptosis. Here it was shown that treatment with the product of the invention and SeR+Se significantly reduced Bcl-2 expression and increased levels of Bax during BPH. These data suggest that the product of the invention treatment inducing expression of Bax in prostate gland, thus stimulating the apoptotic machinery generate an imbalance of Bax/Bcl2 levels

Moreover, these results were also confirmed with the histological evaluation in which the product of the invention treatment, combined of Epilobium with Teoside, was more effective than the other single treatments in preventing the histological features of BPH.

1. A pharmaceutical composition comprising:
an extract of Ajuga reptans comprising an amount of phenylpropanoids from 30% to 70% by weight with respect to the weight of the extract, wherein said amount of phenylpropanoids comprises from 10 to 50% of teupolioside with respect to the total phenylpropanoid weight, and
an extract of Epilobium comprising an amount from 20 to 50% by weight of polyphenols with respect to the weight of the extract, wherein said amount of polyphenols comprises up to 3% by weight of oenothein B with respect to the total weight of polyphenols.

2. The composition according to claim 1 wherein the extract of Ajuga reptans comprises also methylteupolioside and isoteupolioside.

3. The composition according to claim 1, wherein said extract of Ajuga reptans comprises an amount of phenylpropanoids of about 50% by weight with respect to the weight of the extract.

4. The composition according to claim 3, wherein the total amount of phenylpropanoids comprises about 30% by weight of teupolioside with respect to the total amount of phenylpropanoids.

5. The composition according to claim 1, wherein the extract of Ajuga reptans is an extract sold with the commercial name of Teoside™50 by Istituto di Ricerche Biotecnologiche (IRB) S.p.A.

6. The composition according to claim 1, wherein the extract of Epilobium is obtained by Epilobium angustifolium and/or parviflorum and comprises from 20 to 50% of polyphenols, said polyphenols comprising up to 3% of oenothein B.

7. The composition according to claim 1, wherein the extract of Epilobium is an extract of leaves and stems of 50% of Epilobium angustifolium and 50% of Epilobium parviflorum, said extract comprising from 30 to 33% by weight of polyphenols with respect to the weight of the extract, said polyphenols comprising up to 0.4% by weight of oenothein B.

8. The composition according to claim 7, wherein the extract of Epilobium is an extract of leaves and stems of 50% of Epilobium angustifolium and 50% of Epilobium parviflorum, said extract comprising about 31.7% by weight of polyphenols with respect to the weight of the extract, said polyphenols comprising about 0.31% by weight of oenothein B.

9. The pharmaceutical composition according to claim 1, wherein said extract of Ajuga reptans and said extract of Epilobium are in a weight ratio in the range from 1:1 to 1:20.

10. The pharmaceutical composition in accordance with claim 9, wherein said extract of Ajuga reptans and said extract of Epilobium are in a weight ratio in the range from 1:1 to 1:10, preferably of 1:6.

11. A pharmaceutical preparation comprising the composition according to claim 1, and pharmaceutically acceptable excipients.

12. The pharmaceutical preparation in accordance with claim 11, wherein said preparation is a preparation for oral administration.

13. The pharmaceutical preparation in accordance with claim 11, in form of a unitary dose comprising 100-200 mg of Epilobium extract comprising an amount from 20 to 50% by weight of polyphenols with respect to the extract weight, said amount of polyphenols in turn comprising up to 3% of oenothein B, and 10-40 mg of dry extract of Ajuga reptans comprising an amount of polypropanoids of 10-15 mg.

14. The pharmaceutical preparation in accordance with claim 13, in form of a unitary dose comprising 150 mg of Epilobium extract comprising an amount from 20 to 50% of polyphenols with respect to the extract weight, said amount of polyphenols in turn comprising up to 3% of oenothein B, and 25 mg of dry extract of Ajuga reptans comprising an amount of polypropanoids of 12.5 mg.

15. The pharmaceutical preparation in accordance with claim 14, in form of a unitary dose comprising 150 mg of Epilobium extract comprising an amount from 20 to 50% of polyphenols with respect to the extract weight, said amount of polyphenols in turn comprising up to 3% of oenothein B, and 25 mg of dry extract of Ajuga reptans comprising an amount of polypropanoids of 12.5 mg, Zinc-L-pidolate equal to 3 mg of Zinc and sodium selenite equal to 27.5 μg of selenium.

16. A method for the treatment of prostatic diseases, said method comprising:
administering, to a subject having a prostatic disease, the pharmaceutical composition comprising an extract of Ajuga reptans and an extract of Epilobium according to claim 1.

17. The method according to claim 16, wherein said prostatic disease is benign prostatic hyperplasia.
